(19) United States
(12) Patent Application Publication
(10) Pub. No.: US 2019/0343443 A1
(43) Pub. Date: Nov. 14, 2019

(54) STRESS STATE EVALUATION APPARATUS, STRESS STATE EVALUATION SYSTEM, AND NON-TRANSITORY COMPUTER READABLE MEDIUM STORING PROGRAM

(71) Applicant: FUJI XEROX CO., LTD., Tokyo (JP)

(72) Inventor: Kazuhide KOBAYASHI, Kanagawa (JP)

(73) Assignee: FUJI XEROX CO., LTD., Tokyo (JP)

(21) Appl. No.: 16/354,211

(22) Filed: Mar. 15, 2019

(30) Foreign Application Priority Data
May 9, 2018 (JP) 2018-090415

(51) Int. Cl. A61B 5/16; A61B 5/024; A61B 5/00; G16H 10/20; G16H 50/20

(52) U.S. Cl. CPC A61B 5/165; A61B 5/02438; A61B 5/7267; A61B 5/4812; A61B 5/743; A61B 5/6829; G16H 50/20; A61B 2562/0219; A61B 5/6803; A61B 5/6804; A61B 5/681; G16H 10/20

(57) ABSTRACT

A stress state evaluation apparatus includes an acquisition section that acquires living body data from each individual included in a group, a first specification section that specifies a non-stress state of each individual based on the living body data acquired from the acquisition section, an adjustment section that adjusts a determination reference of a stress state of each individual based on the non-stress state of each individual specified in the first specification section, a second specification section that specifies the stress state of each individual using the determination reference adjusted by the adjustment section based on the living body data acquired from the acquisition section, and an operation section that operates a stress state of the group based on the stress state, which is specified by the second specification section, of each individual.

### CROSS-REFERENCE TO RELATED APPLICATIONS

This application is based on and claims priority under 35 USC 119 from Japanese Patent Application No. 2018-090415 filed May 9, 2018.

### BACKGROUND

### (i) Technical Field

The present invention relates to a stress state evaluation apparatus, a stress state evaluation system, and a non-transitory computer readable medium storing a program.

### (ii) Related Art

JP1999-239566A discloses a health management support device includes an input section that is used to input vital data, life style data, and factors, which are used to determine a disease, of an individual, a storage section that stores the data and the factors, which are input by the input section, of the individual, and a display section that displays the data and the factors corresponding to the number of all input people stored in the storage section or the number of a plurality of selected input people in time series.

JP2017-533805A discloses a system including a step of receiving individual stress information with respect to each of a plurality of individuals via a network, and a step of generating a statistic value of stress levels of the plurality of individuals by statistically processing the individual stress information with respect to each of the plurality of individuals in a processing system.

### SUMMARY

In an example according to the above-described related art, visualization of stress through measurement of living body data is taken into consideration. However, there is a person who has a high measured value or a low measured value in peacetime. In a case where data, in which variation in a threshold exists according to the individual, is used as it is, there is a problem in that analysis accuracy is deteriorated.

Here, the stress refers to mental distortion received in the living body.

Aspects of non-limiting embodiments of the present disclosure relate to a stress state evaluation apparatus, a stress state evaluation system, and a non-transitory computer readable medium storing a program, which are capable of evaluating a stress state in a group.

Aspects of certain non-limiting embodiments of the present disclosure overcome the above disadvantages and/or other disadvantages not described above. However, aspects of the non-limiting embodiments are not required to overcome the disadvantages described above, and aspects of the non-limiting embodiments of the present disclosure may not overcome any of the disadvantages described above.

According to an aspect of the present disclosure, there is provided a stress state evaluation apparatus including: an acquisition section that acquires living body data from each individual included in a group; a first specification section that specifies a non-stress state of each individual based on the living body data acquired from the acquisition section; an adjustment section that adjusts a determination reference of a stress state of each individual based on the non-stress state of each individual specified in the first specification section; a second specification section that specifies the stress state of each individual using the determination reference adjusted by the adjustment section based on the living body data acquired from the acquisition section; and an operation section that operates a stress state of the group based on the stress state, which is specified by the second specification section, of each individual.

Here, the stress state refers to a state in which stress exists, and the non-stress state refers to a state in which the stress does not exist. The non-stress state includes a state (during sleep) which is insensitive to any stress as in a case of during sleep, and a state (in peacetime) which is insensitive to the stress because regular duties are performed.

### BRIEF DESCRIPTION OF THE DRAWINGS

Exemplary embodiment(s) of the present invention will be described in detail based on the following figures, wherein:

FIG. 1 is a configuration diagram illustrating a stress state evaluation system according to an exemplary embodiment of the present invention;

FIG. 2 is a block diagram illustrating hardware of a control unit of a server used in the exemplary embodiment of the present invention;

FIG. 3 is a block diagram illustrating a function of the control unit of the server used in the exemplary embodiment of the present invention;

FIG. 4 is an explanatory diagram illustrating a display example of a smart phone used in the exemplary embodiment of the present invention;

FIG. 5 is flowchart illustrating a control flow of the server used in the exemplary embodiment of the present invention;

FIG. 6 is a chart illustrating calculated stress values for respective individuals in the exemplary embodiment of the present invention;

FIG. 7 is a chart illustrating the number of people in a stress state in the exemplary embodiment of the present invention;

FIG. 8 is a chart illustrating results acquired by analyzing and accumulating individual stresses for respective individuals in the exemplary embodiment of the present invention;

FIG. 9 is a chart illustrating calculated stress values of a group by units of date in the exemplary embodiment of the present invention;

FIG. 10 is a chart illustrating the stresses of the group for the past one month as averages for respective days in the exemplary embodiment of the present invention;

FIG. 11 is a chart illustrating timing, at which a notification of the number of people in the stress state is provided, in the exemplary embodiment of the present invention;

and

FIG. 12 is a chart illustrating notification timing in a case where the calculated values of the stresses of the group are counted by units of date in the exemplary embodiment of the present invention.

### DETAILED DESCRIPTION

Subsequently, an exemplary embodiment of the present invention will be described in detail with reference to the accompanying drawings.

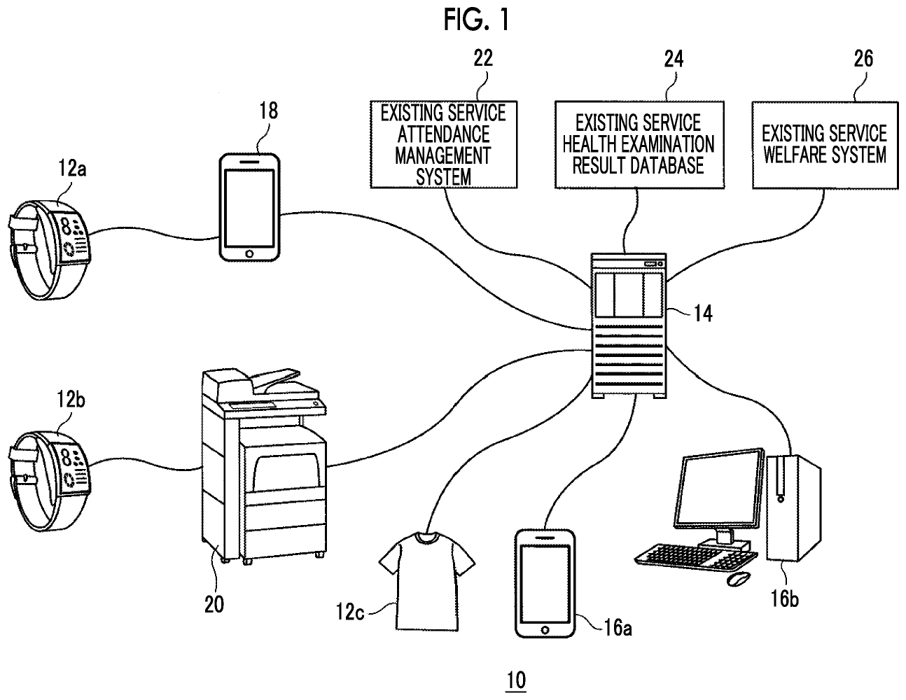

FIG. 1 illustrates a stress state evaluation system 10 according to the exemplary embodiment of the present invention.

The stress state evaluation system 10 includes a plurality of detection devices 12a, 12b, and 12c, a server 14 which is connected to the detection devices, and display devices 16a and 16b which display an output from the server 14.

The plurality of detection devices 12a, 12b, and 12c are wearable terminals (terminals which are worn by an individual). The wearable terminals 12a, 12b, and 12c may detect living body data, and may include a heart rate meter (which may be a pulsimeter) which detects heart rate fluctuations in an individual and an accelerometer which detects acceleration. In the exemplary embodiment, the wearable terminals 12a and 12b are watch types and the wearable terminal 12c is a clothes type. In addition thereto, the wearable terminals 12a, 12b, and 12c include a band type to be wound around a wrist or an ankle, a spectacle type, a cap type, an adhesion patch type, and the like. The wearable terminals 12a and 12b transmit the detected living body data to the smart phone 18 or an image forming apparatus 20 via, for example, Bluetooth (which is one of short-distance radio communication standards and is a registered trademark). The living body data is transmitted to the server 14 from the smart phone 18 and the image forming apparatus 20 in a wireless or wired manner or via the Internet. In addition, the wearable terminal 12c transmits the living body data to the server 14 through, for example, Wi-Fi (which is one type of wireless LAN and is a registered trademark).

The server 14 may perform health management of a group, such as employees of a company, in association with an existing system or a database. For example, the server 14 may acquire data relevant to the employees from a attendance management system. 22 which is used to manage attendance of the employees, a health examination result database 24 which stores health examination results of the employees, and a welfare system 26 which is used to manage welfare of the employees, and may grasp a relation with a stress state.

The server 14 acquires the living body data from the wearable terminals 12a, 12b, and 12c, evaluates stress states of the individual and the group based on the living body data, notifies, for example, the display device 16a, which is an individual smart phone, of evaluated results as information or notifies the display device 16b, which is a personal computer of a manager, of the information.

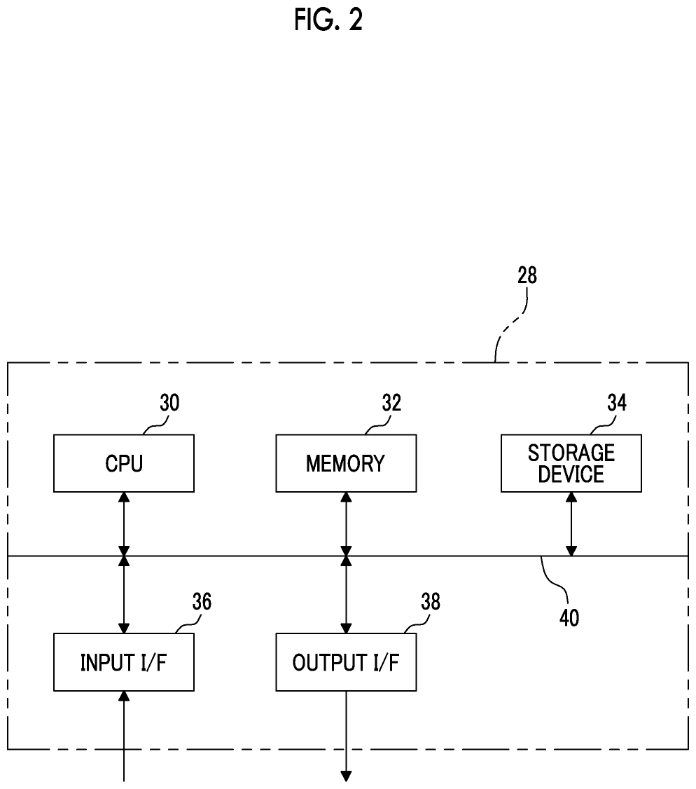

FIG. 2 illustrates a hardware configuration of a control unit 28 in the server 14.

The control unit 28 includes a CPU 30, a memory 32, a storage device 34, an input interface 36, and an output interface 38. The CPU 30, the memory 32, the storage device 34, the input interface 36, and the output interface 38 are connected to each other via a control bus 40.

The CPU 30 performs a predetermined process based on the control program stored in the memory 32. The data is input to the input interface 36 via a network or the like. The output interface 38 outputs the data toward the network or the like. The storage device 34 includes, for example, a hard disk or the like.

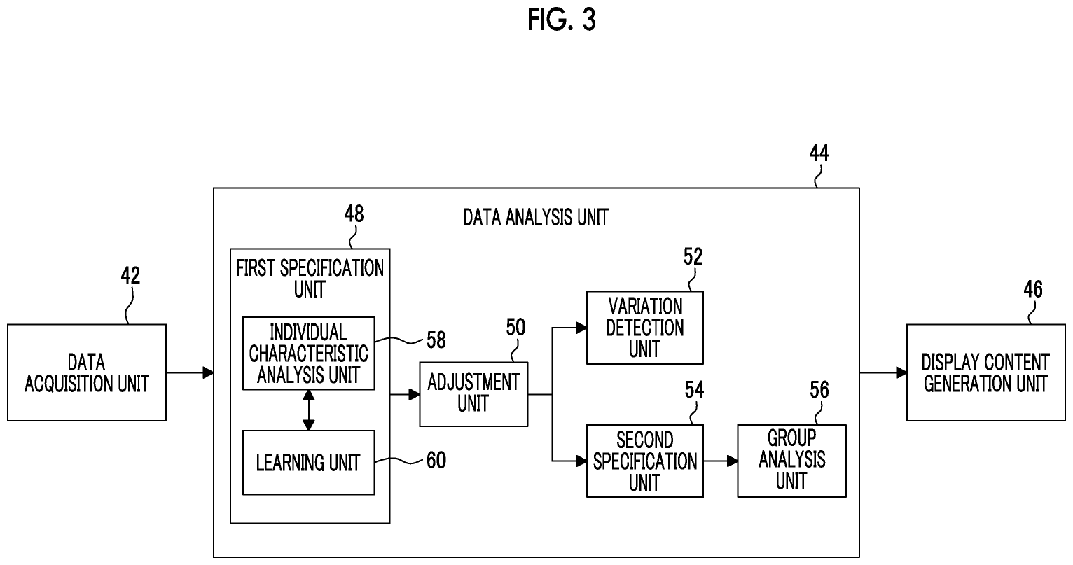

FIG. 3 is a functional block diagram illustrating the control unit 28 in the server 14.

A data acquisition unit 42 acquires the living body data via the above-described input interface 36. A data analysis unit 44 analyzes the living body data acquired in the data acquisition unit 42. A display content generation unit 46, which is included in an output section, generates content to be displayed based on a result acquired through analysis in the data analysis unit 44, and outputs the content.

The data analysis unit 44 includes a first specification unit 48, an adjustment unit 50, a variation detection unit 52, a second specification unit 54, and a group analysis unit 56. The first specification unit 48 includes an individual characteristic analysis unit 58 and a learning unit 60. The individual characteristic analysis unit 58 performs power spectrum analysis on time-series frequency components at R-R intervals based on heart rate variation acquired by, for example, the data acquisition unit 42. In the time-series frequency components at the R-R intervals, the individual characteristic analysis unit 58 acquires Low Frequency (LF) components affected by a sympathetic nerve activity, High Frequency (HF) components affected by parasympathetic nerves, and, further, a sympathetic nerve activity index (LF/HF). The sympathetic nerve activity index (LF/HF) is used to determine a non-stress state or the stress state.

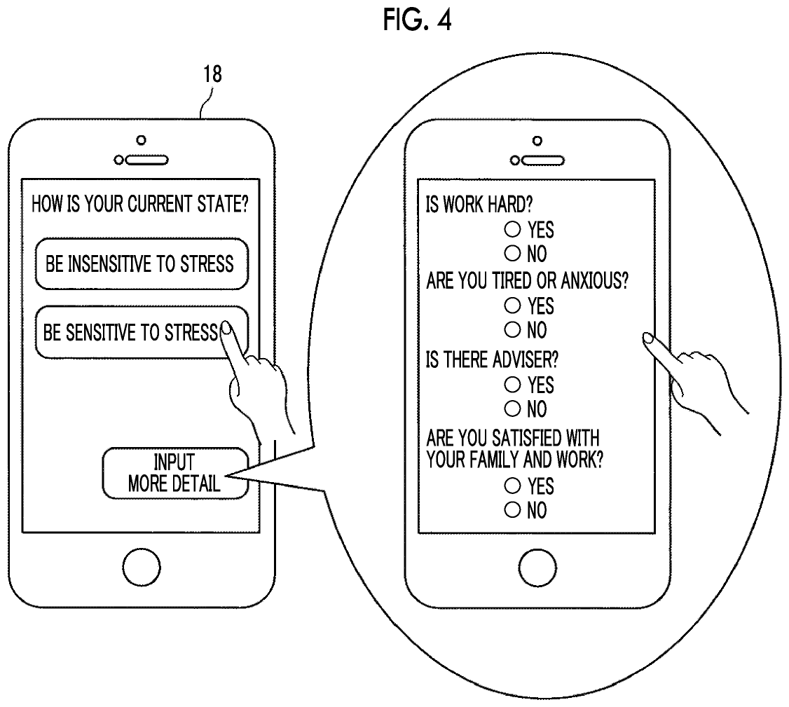

The learning unit 60 performs learning based on, for example, a result acquired by performing stress state investigation. For example, as illustrated in FIG. 4, in the stress state investigation, a questionnaire is transmitted to the smart phone 18 of each individual. The questionnaire may simply include selection between “be insensitive to the stress” or “be sensitive to the stress”, or, more specifically, may include questions, such as “is work hard?”, “are you tired or anxious?”, “is there an adviser?”, and “are you satisfied with your family and work?”.

The learning unit 60 learns results of the stress state investigation as teaching data. The stress state investigation may be performed for each predetermined period, or may be performed in a case where the sympathetic nerve activity index (LF/HF) increases rather than a predetermined value. Newly received results of the stress state investigation are retroactively reflected.

In addition, a stress state acquired during sleeping may be referred to. With regard to sleeping or non-sleeping, for example, it is possible to set a state of seldom movement in outputs from the acceleration sensors, which are mounted on the detection devices 12a, 12b, and 12c, as a case of sleeping. In addition, instead of the stress state investigation, it is possible to determine an output acquired in the case of sleeping as the non-stress state.

The adjustment unit 50 adjusts a determination reference of whether the non-stress state or the stress state based on the non-stress state, which is specified in the first specification unit 48, of each individual. There is a large individual difference in whether or not be sensitive to the stress. Therefore, it is demanded to adjust the determination reference for each individual.

The variation detection unit 52 detects a variation from the non-stress state. The detection is performed in such a way that the variation from the non-stress state and a way of the variation are detected according to the determination reference adjusted in the adjustment unit 50. For example, the variation from the non-stress state is detected in a case where a state in the parasympathetic nerve activity is dominant changes to a state in which the sympathetic nerve activity is dominant.

In a case where the variation detection unit 52 detects the stress state, the second specification unit 54 specifies that the individual is in the stress state.

The group analysis unit 56 collects a variation in the stress of each individual using the variation detection unit 52, and analyzes a change as the group. The group analysis unit 56 performs adjustment such that the stress state, which is detected in the variation detection unit 52, of each individual is collected for each characteristic of each individual in the group. For example, an individual, in which the sympathetic nerve activity is dominant from the non-stress state, is biased to suppress the stress low, and an individual, in which the parasympathetic nerve activity is dominant from the non-stress state, is biased to increase the stress.

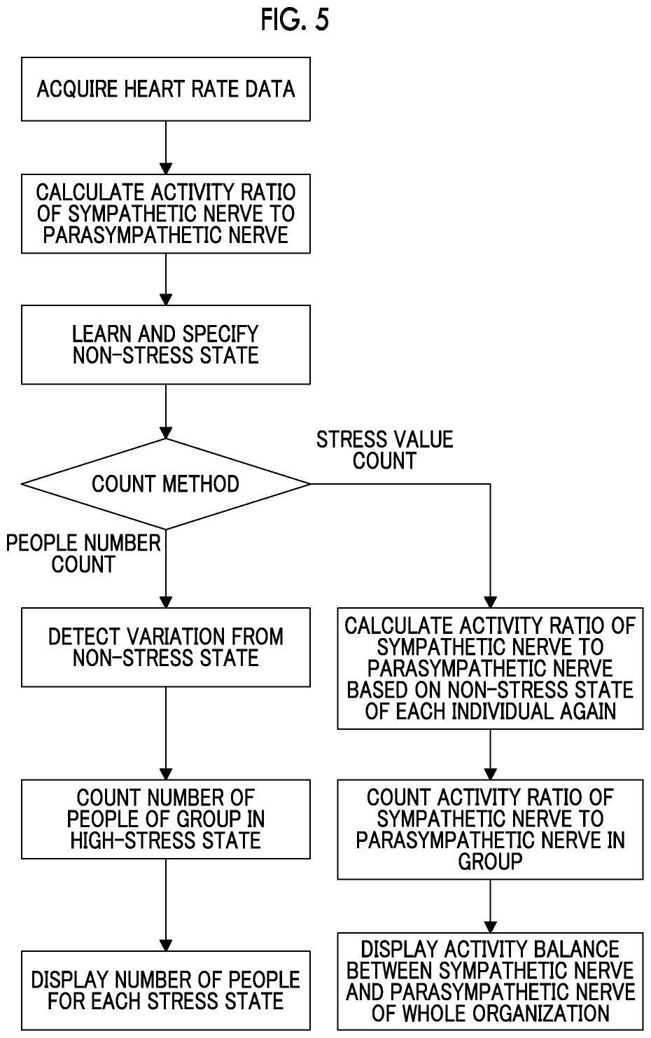

FIG. 5 is a flowchart illustrating a control operation in the server 14.

First, in step S10, heart rate data is acquired. In subsequent step S12, an activity ratio of the sympathetic nerve to the parasympathetic nerve is calculated based on the heart rate data acquired in step S10. In subsequent step S14, the non-stress state is specified through the learning based on the results of the stress state investigation and behaviors of the individual.

In subsequent step S16, a count method is determined. The count method includes a people number count and a stress value count. The count method is determined by, for example, an instruction from a manager.

In a case where it is determined to be the people number count in step S16, the process proceeds to step S18, and the variation from the non-stress state is detected. In subsequent step S20, the number of people of the group in a high-stress state is counted. In subsequent step S22, output is performed to display the number of people for each stress state.

In contrast, in a case where it is determined to be the stress value count in step S16, the process proceeds to step S24, and the activity ratio of the sympathetic nerve to the parasympathetic nerve is calculated again based on the non-stress state of each individual again. In subsequent step S26, the activity ratio of the sympathetic nerve to the parasympathetic nerve in the group is counted. In subsequent step S28, output is performed to display an activity balance (intensity of the stress state) between the sympathetic nerve and the parasympathetic nerve of whole organization.

Subsequently, a display example will be described.

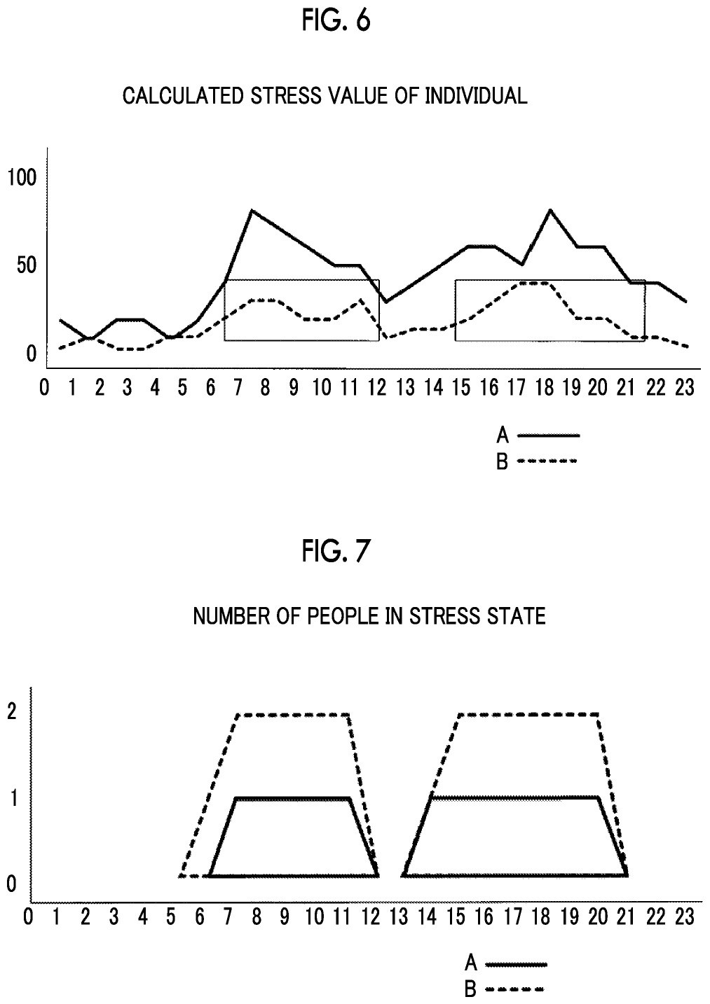

FIG. 6 illustrates transition of calculated stress values for respective individuals.

Here, a horizontal axis indicates time of a day, and a vertical axis indicates a relative value corresponding to the sympathetic nerve activity index (LF/HF).

In a case where the number of people corresponding to the individuals in the stress state is displayed and a determination reference (threshold) of whether the non-stress state or the stress state is, for example, evenly set to 40, an individual A is in the stress state and the individual B is in the non-stress state during, for example, half past six to twelve o'clock. Here, the number of people corresponding to the individuals in the stress state in the group is one.

However, the individual B is in the stress state, and thus erroneous determination is performed. Here, in a case where it is assumed that a threshold of the individual B is approximately 20, it is grasped that the individual B is also in the stress state, and thus the individual B is also included in the number of people in the stress state, as illustrated in a first example of FIG. 7.

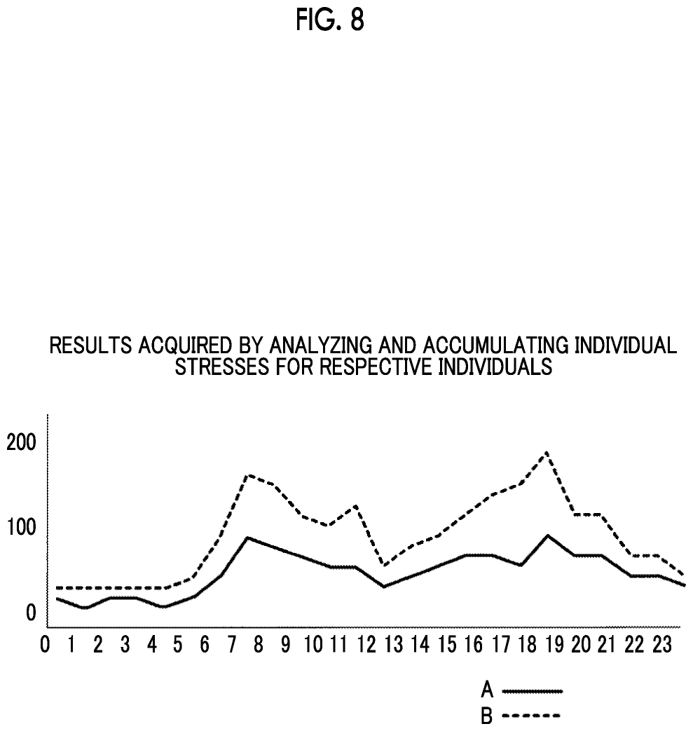

FIG. 8 is a second example and illustrates results acquired by analyzing and accumulating individual stresses for respective individuals. In the example, although the individual A is only biased by, for example, approximately 20%, the individual B is biased by, for example, approximately 400%, and thus the balance is maintained such that there is no difference between the individuals.

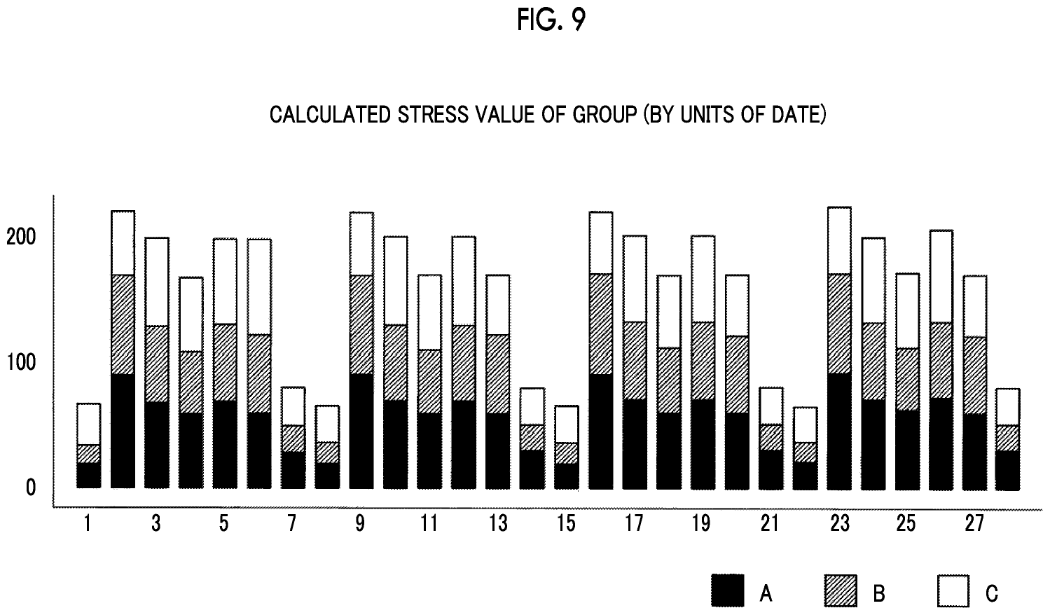

FIG. 9 is a third example and illustrates the stress values of a group which includes the individual A, the individual B, and an individual C by units of date. Here, the individual difference between the individuals A to C is taken into consideration, and the individual difference is adjusted and accumulated according to the non-stress state. A time axis of display may indicate units of a week or a month, and may be freely changed.

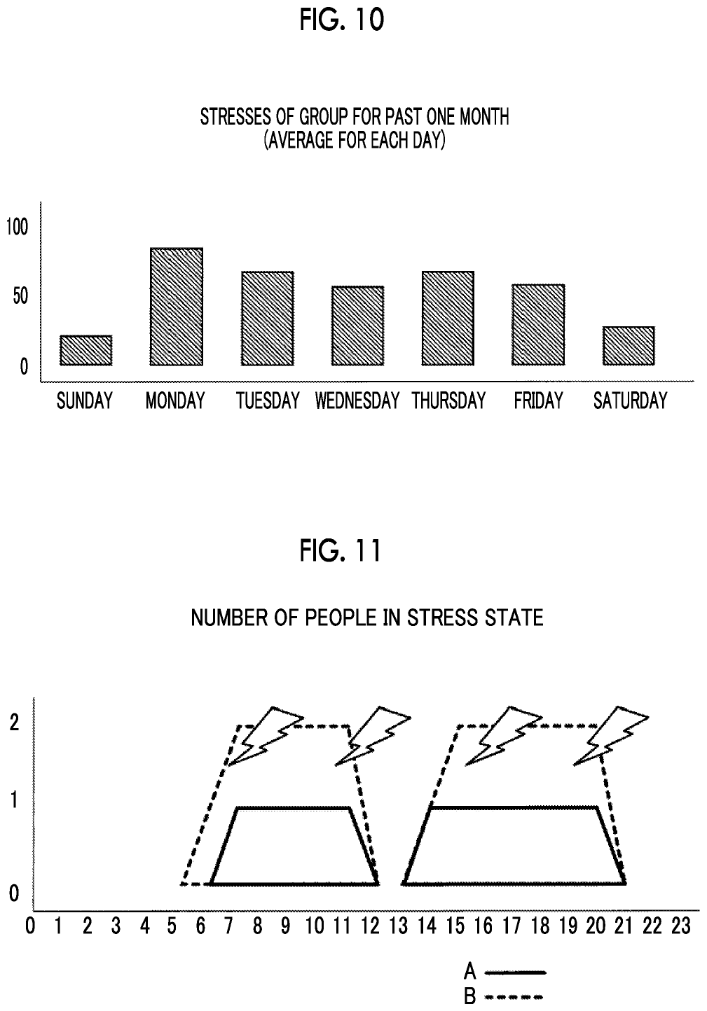

FIG. 10 illustrates the stresses of the group for past one month and illustrates averages on respective days. For example, it is understood that stresses of the group are high on Monday. The stresses may be displayed according to each date, each time zone, or the like.

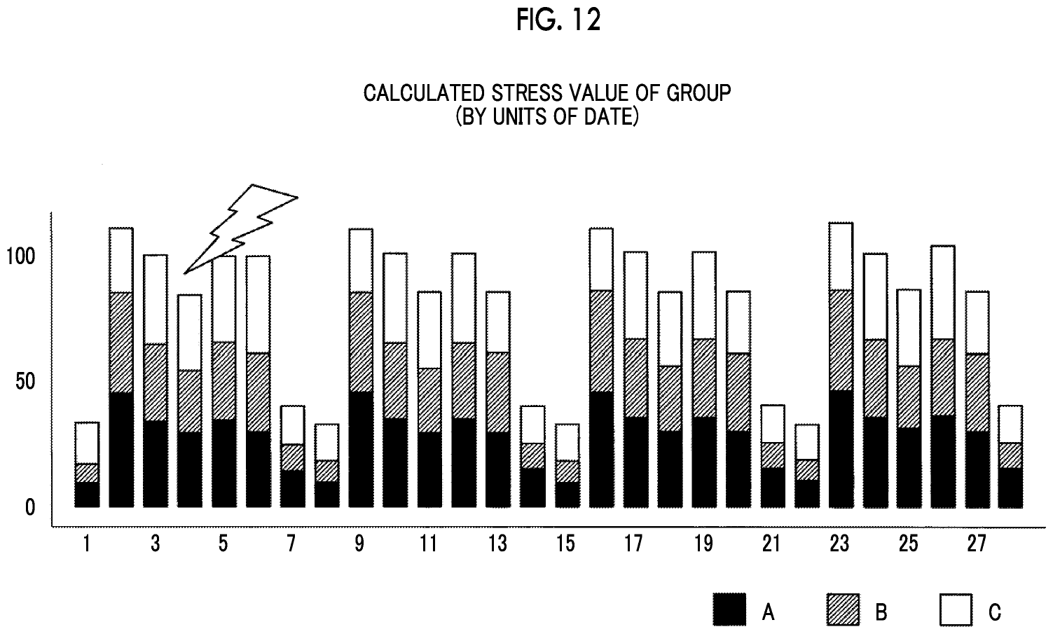

FIGS. 11 and 12 illustrate timing at which a notification is provided to the manager. For example, in FIG. 11, the notification is provided at time in which a change to a stress state of the group occurs. In addition, in FIG. 12, the notification is provided at time in which the stress state is continued. For example, the notification is provided in a case where the stress state exceeds a predetermined value for three consecutive days.

The foregoing description of the exemplary embodiments of the present invention has been provided for the purposes of illustration and description. It is not intended to be exhaustive or to limit the invention to the precise forms disclosed. Obviously, many modifications and variations will be apparent to practitioners skilled in the art. The embodiments were chosen and described in order to best explain the principles of the invention and its practical applications, thereby enabling others skilled in the art to understand the invention for various embodiments and with the various modifications as are suited to the particular use contemplated. It is intended that the scope of the invention be defined by the following claims and their equivalents.

What is claimed is:

1. A stress state evaluation apparatus comprising:
an acquisition section that acquires living body data from each individual included in a group;
a first specification section that specifies a non-stress state of each individual based on the living body data acquired from the acquisition section;
an adjustment section that adjusts a determination reference of a stress state of each individual based on the non-stress state of each individual specified in the first specification section;
a second specification section that specifies the stress state of each individual using the determination reference adjusted by the adjustment section based on the living body data acquired from the acquisition section; and
an operation section that operates a stress state of the group based on the stress state, which is specified by the second specification section, of each individual.

2. The stress state evaluation apparatus according to claim 1,
wherein the first specification section specifies the non-stress state based on a result acquired by performing a stress state investigation.

3. The stress state evaluation apparatus according to claim 2,
wherein the first specification section specifies the non-stress state based on the result acquired by performing the stress state investigation for each predetermined period.

4. The stress state evaluation apparatus according to claim 2,
wherein the first specification section specifies the non-stress state based on the result acquired by performing the stress state investigation in a case where the living body data acquired by the acquisition section changes.

5. The stress state evaluation apparatus according to claim 2,
wherein the first specification section specifies the non-stress state by learning the result acquired by performing the stress state investigation.

6. The stress state evaluation apparatus according to claim 3,
wherein the first specification section specifies the result acquired by performing the stress state investigation and specifies the non-stress state.

7. The stress state evaluation apparatus according to claim 4,
wherein the first specification section specifies the result acquired by performing the stress state investigation and specifies the non-stress state.

8. The stress state evaluation apparatus according to claim 2,
wherein the first specification section specifies the non-stress state with the living body data which is acquired from the acquisition section while the individual is sleeping.

9. The stress state evaluation apparatus according to claim 1, further comprising:
an output section that outputs an operation result acquired through an operation performed by the operation section.

10. The stress state evaluation apparatus according to claim 2, further comprising:
an output section that outputs an operation result acquired through an operation performed by the operation section.

11. The stress state evaluation apparatus according to claim 3, further comprising:
an output section that outputs an operation result acquired through an operation performed by the operation section.

12. The stress state evaluation apparatus according to claim 4, further comprising:
an output section that outputs an operation result acquired through an operation performed by the operation section.

13. The stress state evaluation apparatus according to claim 5, further comprising:
an output section that outputs an operation result acquired through an operation performed by the operation section.

14. The stress state evaluation apparatus according to claim 6, further comprising:
an output section that outputs an operation result acquired through an operation performed by the operation section.

15. The stress state evaluation apparatus according to claim 9,
wherein the output section performs output to display the operation result.

16. The stress state evaluation apparatus according to claim 15,
wherein the output section performs output to display the number of people in the stress state in the group.

17. The stress state evaluation apparatus according to claim 15,
wherein the output section performs output to display an intensity of the stress state in the group.

18. The stress state evaluation apparatus according to claim 15,
wherein the output section performs output to display a time-series graph.

19. A stress state evaluation system comprising:
a detection device that is mounted on each individual included in a group and detects living body data;
a server that is connected to the detection device; and
a display device that displays an output from the server,
wherein the server includes
an acquisition section that acquires the living body data from the detection device,
a first specification section that specifies a non-stress state of each individual based on the living body data acquired from the acquisition section,
an adjustment section that adjusts a determination reference of a stress state of each individual based on the non-stress state of each individual acquired from the first specification section,
a second specification section that specifies the stress state of each individual using the determination reference adjusted by the adjustment section based on the living body data acquired from the acquisition section,
an operation section that operates a stress state of the group based on the stress state, which is specified by the second specification section, of each individual, and
an output section that outputs an operation result of the operation section to be displayed on the display device.

20. A non-transitory computer readable medium storing a program causing a computer to perform a process comprising:
acquiring living body data from each individual included in a group;
specifying a non-stress state of each individual based on the acquired living body data;
adjusting a determination reference of a stress state of each individual based on the non-stress state of each specified individual;
specifying the stress state of each individual using the adjusted determination reference; and
operating a stress state of the group based on the specified stress state of each individual.
